(19) United States
(12) Patent Application Publication
(10) Pub. No.: US 2012/0321656 A1
(43) Pub. Date: Dec. 20, 2012

(54) OLIGOPEPTIDES AND THEIR USE FOR TREATING INFECTIOUS DISEASES

(75) Inventors: MATTHIAS W. RATH, APTOS, CA (US); ALEKSANDRA NIEDZWIECKI, APTOS, CA (US)

(73) Assignee: MATTHIAS RATH, APTOS, CA (US)

(21) Appl. No.: 13/525,005

(22) Filed: Jun. 15, 2012

Related U.S. Application Data
(60) Provisional application No. 61/498,657, filed on Jun. 20, 2011.

(51) Int. Cl. A61K 39/04; A61K 39/39; A61P 31/06

(52) U.S. Cl. 424/190.1; 424/248.1

(57) ABSTRACT

The invention discloses identification, method of making and therapeutic use of synthetic oligopeptides for the treatment of infectious diseases, in particular tuberculosis. The oligopeptides are designed using virulence mediating protein for Mycobacterium Tuberculosis bacteria. The antibodies may be used for diagnostic and treatment purposes of infectious diseases. In particular, the sequences with SEQ ID 1 to 11 may be used to produce such oligopeptides synthetically. Suppression of activity of mycobacterium tuberculosis may be achieved with oligopeptides analogous to SEQ ID 1 to 11 as a therapeutic drug and/or as a vaccine in a mammal.

### CROSS REFERENCE TO RELATED APPLICATION

The instant application is a continuation-in-part application and claims priority to pending U.S. Provisional patent application 61/498,657, filed on 20 Jun., 2011. The disclosure is hereby incorporated by this reference in its entirety for all of its teachings. This application contains sequence listing that has been submitted as an ASCII file named RIPLLC018014US1sequence_ST25, the date of creation Jun. 12, 2012, and the size of the ASCII text file in bytes is 3 kb.

### FIELD OF TECHNOLOGY

This disclosure relates generally to designing and utilizing novel oligopeptide sequences to be used as therapeutic agents for treating infectious diseases. More specifically, this disclosure relates to using the oligopeptide as a vaccine to treat tuberculosis.

### BACKGROUND OF THE INVENTION

Mycobacteria (MB) cause a range of infectious diseases in animals and humans. Among the most virulent diseases in man caused by this species of bacteria are tuberculosis (TB) and leprosy. TB remains one of the world's leading causes of death caused by a single pathogen. According to the World Health Organization (WHO), every year 3 million people worldwide die from TB and 10 million new cases of infection with the virulent Mycobacterium Tuberculosis (MBT) occur.

Over the past two decades, TB has been resurging worldwide and in some countries, especially in the developing world, this epidemic is increasing at a dramatic rate. Contributing to this development is the acquired immune deficiency syndrome (AIDS) epidemic with co-infection of MBT being one of the hallmarks of advanced stages of AIDS.

Almost one hundred years after the introduction of the BCG vaccine—still the most widely used vaccine—today, the epidemic continues. The efficacy of BCG has been unsatisfactory while undesired side-effects associated with this vaccine are not uncommon.

Because of the controversial efficacy of BCG vaccination, public health policies are increasingly relying on pharmaceutical approaches. Due to the nature of the MBT infection and its survival within macrophages, conventional antibiotics show a limited effect. The most widely used drugs are isoniazide, rifampicin, pyrazinamide, fluoroqionolones and other chemotherapeutic drugs. These highly toxic drugs cause severe side effects, including irreversible damage to the liver and other organs, anemia, drug-induced immune deficiencies and death.

Considering the fact that these anti-TB drugs are also given to tens of thousands of children world-wide with devastating consequences for their physical development and future health there exists an urgent need for the development of an effective, safe and affordable therapy in fighting TB infections in humans.

### SUMMARY

The current application discloses a sequence and a composition of sequences with SEQ ID 1 to 11 oligopeptides and a method of using the same as a vaccine to treat infectious disease.

In one embodiment, oligopeptide analogs for MT Heparin-Binding Hemagglutinin, MT Antigen 85 (Ag 85), MT Exported Repetitive Protein (Erp), MT Cytotoxin/Hemolysin are designed and synthesized. In another embodiment, these oligopeptides are formulated to be used as a vaccine.

In one embodiment, the following oligopeptide sequences from Mycobacterium Tuberculosis were used to produce a vaccine.

MT Heparin-Binding Hemagglutinin:

SEQ ID 1-T-D-T-R-S-R-V-E-E-S-R-A-R-L-
SEQ ID 2-S-R-Y-N-E-L-V-E-R-G-E-A

MT Antigen 85 (Ag 85):

SEQ ID 3-S-M-G-R-D-I-K-V-Q-F-Q-G-
SEQ ID 4-L-R-A-Q-D-D-Y-N-G-W-D-I-
SEQ ID 5-T-Y-K-W-E-T-F-L-T-R-E-M-P-A-
SEQ ID 6-S-D-P-A-W-K-R-N-D-P-M-V-Q-I-P-R-L-
SEQ ID 7-G-D-N-I-P-A-K-F-L-E-G-T-L-R-T-

MT Exported Repetitive Protein (Erp):

SEQ ID 8-V-Y-E-S-T-E-T-T-E-R-P-E-H-H-E-F-K-Q-A-
SEQ ID 9-N-E-L-G-A-S-Q-A-I-D-L-L-K-G-V-

MT Cytotoxin/Hemolysin:

SEQ ID 10-T-D-S-E-R-A-W-V-S-R-G-A-
SEQ ID 11-A-G-R-R-C-L-D-A-G

In one embodiment, the sequence of oligopeptide may be modified, but is not limited, using at least one of the following modifications such as a mutation, deletion, substitution, addition at the c-terminal or N-terminal end, and/or a combination of any of these modifications and used as a vaccine to treat infectious diseases.

In one embodiment, the oligopeptide as shown in sequences with SEQ ID 1 to 11 may be used as a vaccine. In another embodiment, oligopeptide as shown in sequences with SEQ ID 1 to 11 may be used in combination with any one of the other sequences with SEQ ID 1 to 11 of oligopeptide as a vaccine. In another embodiment, all oligopeptide as shown in sequences with SEQ ID 1 to 11 may be combined to produce a vaccine.

The oligopeptide sequences, in one embodiment, may be either linear or circular in design. In another embodiment, the oligopeptide may be a repeat of these oligopeptide sequences.

In another embodiment, the oligopeptide may have either haptens or polyglycans attached to them for efficient delivery. In another embodiment, an adjuvant may be added. In another embodiment, a suitable pharmaceutically acceptable carrier may be used to form the vaccine from oligopeptide as shown in sequences with SEQ ID 1 to 11.

In another embodiment, the oligopeptide as shown in sequences with SEQ ID 1 to 11 may have simultaneous mutation, deletion, substitution, addition at the c-terminal or N-terminal end, and/or a combination of any of these modifications and used as a vaccine to treat infectious diseases. In another embodiment a mammal may be vaccinated to prevent and/or treat the infectious disease such as Tuberculosis. In another embodiment, a mammal may be vaccinated to induce immune response with virulence mediated protein, wherein the virulence mediating protein are at least one of a MT Heparin binding hemagglutinin, MT Antigen 85, MT exported repetitive protein and MT cytotoxin/hemolysin protein.

In one embodiment, the virulence mediating proteins MT Heparin binding hemagglutinin, MT Antigen 85, MT exported repetitive protein and MT cytotoxin/hemolysin protein comprises of SEQ ID 1 to 11.

In one embodiment, a composition for an oligopeptide as a vaccine comprising of oligopeptide as shown in sequences with SEQ ID 1 to 11 individually or combination thereof.

In one embodiment the therapeutically effective amount may be rendered, but not limited to, as an injection. Other embodiments may include peroral, topical, transmucosal, inhalation, targeted delivery and sustained release formulations. Some examples may be an aerosol, spray for inhalation, rectally as suppositories, tablet with preferred coating to enhance absorption and prevent premature delivery in the digestive tract.

The vaccine may be produced for inoculating human being as a preventive measure or treatment method or curative measure. The vaccine using oligopeptide as shown in sequences with SEQ ID 1 to 11 is used for human being to treat TB. The human may use the vaccine as prevention or as a treatment after contracting the disease.

The composition, method, and treatment disclosed herein may be implemented in any means for achieving various aspects, and may be executed in a form suitable for the mammal.

### DETAILED DESCRIPTION

Several sequences and methods for immunizing and treating TB using oligopeptide as shown in sequences with SEQ ID 1 to 11 as a vaccine are described herein. Although the present embodiments have been described with reference to specific example embodiments, it will be evident that various modifications and changes may be made to these embodiments without departing from the broader spirit and scope of the various embodiments.

Mycobacteria cause a range of infectious diseases in animals and humans. Among the most virulent diseases in man caused by this species of bacteria are TB and leprosy. TB infections are caused by Mycobacterium Tuberculosis (MBT). The most common path of infection by MBT bacteria is through airways. In the lung, these pathogens are taken up by alveolar macrophages. Due to special defense mechanisms, MBT is able to escape phagocytosis by the macrophage and is able to survive and multiply within these cells.

Almost a century after the introduction of the BCG vaccine, the tuberculosis (TB) epidemic continues almost unabated and the need for new, effective, safe and affordable vaccines is a global health challenge. BCG is prepared from an attenuated live strain of bovine mycobacterium bovis, a fact that explains both its unsatisfactory efficacy against human mycobacterium tuberculosis (MBT) as well as its frequent side-effects. The present invention describes the identification of oligopeptides from virulence mediating proteins of MBT which were shown to function as epitopes for antibody production and thus are candidates for effective vaccines against human tuberculosis.

In most cases, MBT infections occur in a latent forms without the development of TB disease. In other cases, the TB disease openly manifests itself within the lungs or by spreading via the blood stream and lymph system to other organs. In this way, TB disease can affect joints, bones, the central nervous system, the genital and urinary tract, intestine, skin and other organs.

New therapeutic targets have to take the specific structure of the MBT into account. The instant application has the identified certain proteins of the MBT that are associated with mediating virulence. These virulence-mediating proteins can generally be divided into categories of membrane proteins of MBT and proteins that are secreted and exported by MBT into the infected cells and/or tissue.

Virulence-Mediating Proteins

In this disclosure we analyzed the amino acid sequences of several virulence-mediating proteins from MBT. they are

| | |
|---|---|
| a) MBT Heparin-Binding Hemagglutinin | (SEQ ID 1-2) |
| b) MBT Antigen 85 (Ag 85) | (SEQ ID 3-7) |
| c) MBT Exported Repetitive Protein (Erp) | (SEQ ID 8-9) |
| d) MBT Cytotoxin/Hemolysin | (SEQ ID 10-11) |

Use in Prevention and Treatment of Tuberculosis

Synthetic analogs of the identified signal oligopeptides of these virulence-mediating MT proteins can be used to inhibit their biological effect and thereby block TB infections. These therapeutic agents as vaccines or medication can be used in the prevention as well as the treatment of TB infections.

Used as vaccines, the synthetic analogs to these signal oligopeptides can be used to raise antibodies against these antigenic oligopeptides. In the instant application, the antibodies developed against the vaccine would also inhibit the interaction of the virulence-mediating MBT proteins with host cells or tissue, thereby blocking TB infection.

Increase of Therapeutic Efficacy

To increase the therapeutic effect of these synthetic analogs to epitopes of MBT virulence-mediating proteins, the claimed sequences can be modified in the following way:

a) substitutions of one or several amino acids with other amino acids

b) insertion of one or several amino acids

c) deletions of one or several amino acids

d) additions of one or several amino acids at the C-terminal or N-terminal end

e) use of the synthetic oligopeptide in linear form

f) use of the synthetic oligopeptide in circular form

g) use of adjuvants

h) use of pharmaceutically accepted carriers

This would enable to design the treatment method for genetic variations, personalized medicine and age appropriate dosage regimen. The treatment method may be administered once, repeatedly or as a regular prescription depending on the state of the disease and physicians recommendations.

Therapeutic Delivery Paths

Synthetic oligopeptides based on the virulence mediating proteins such as wherein the virulence mediating protein are at least one of a MT Heparin binding hemagglutinin, MT Antigen 85, MT exported repetitive protein and MT cytotoxin/hemolysin protein comprising of sequences SEQ ID 1 to 11 as claimed in the instant application can be therapeutically applied to patients:
a) parenterally, i.e. intravenously
b) subcutaneously
c) intramuscularly
d) transdermally
e) as a spray or aerosol, e.g. for inhalation
f) rectally, e.g. as suppositories
g) orally, eg. with specific coating to prevent premature digestion of these oligopeptide in the intestine

Another way to administer the oligopeptides is via a replication-deficient virus (e.g. adenovirus type 35) expressing one or more of the oligopeptides from Seq. ID 1 to 11.

Drug formulations suitable for these administration routes can be produced by adding one or more pharmacologically acceptable carriers to the agent and then treating the mixture through a routine process known to those skilled in the art. The mode of administration includes, but is not limited to, non-invasive peroral, topical (example transdermal), enteral, transmucosal, targeted delivery, sustained release delivery, delayed release, pulsed release and parenteral methods. Peroral administration may be administered both in liquid and dry state.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition as an active ingredient. Subject compositions may also be administered as a bolus, electuary, or paste.

When an oral solid drug product is prepared, oligopeptide sequence of sequences with SEQ ID 1 to 11 is mixed with an excipient (and, if necessary, one or more additives such as a binder, a disintegrant, a lubricant, a coloring agent, a sweetening agent, and a flavoring agent), and the resultant mixture is processed through a routine method, to thereby produce an oral solid drug product such as tablets, coated tablets, granules, powder, or capsules. Additives may be those generally employed in the art. Examples of the excipient include lactate, sucrose, sodium chloride, glucose, starch, calcium carbonate, kaolin, microcrystalline cellulose, and silicic acid; examples of the binder include water, ethanol, propanol, simple syrup, glucose solution, starch solution, liquefied gelatin, carboxymethylcellulose, hydroxypropylcellulose, hydroxypropyl starch, methyl cellulose, ethyl cellulose, shellac, calcium phosphate, and polyvinyl pyrrolidone; examples of the disintegrant include dried starch, sodium arginate, powdered agar, sodium hydrogencarbonate, calcium carbonate, sodium lauryl sulfate, monoglyceryl stearate, and lactose; examples of the lubricant include purified talc, stearic acid salts, borax, and polyethylene glycol; and examples of the sweetening agent include sucrose, orange peel, citric acid, and tartaric acid.

When a liquid drug product for oral administration is prepared, oligopeptide sequence of sequences with SEQ ID 1 to 11 is mixed with an additive such as a sweetening agent, a buffer, a stabilizer, or a flavoring agent, and the resultant mixture is processed through a routine method, to thereby produce an orally administered liquid drug product such as an internal solution medicine, syrup, or elixir. Examples of the sweetening agent include vanillin; examples of the buffer include sodium citrate; and examples of the stabilizer include tragacanth, acacia, and gelatin.

For purposes of transdermal (e.g., topical) administration, dilute sterile, aqueous or partially aqueous solutions (usually in about 0.1% to 5% concentration), otherwise similar to the above parenteral solutions, may be prepared.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated compound(s) and composition(s). Formulations which are suitable for vaginal administration also include pessaries, tampons, creams, gels, pastes, foams, or spray formulations containing such carriers as are known in the art to be appropriate.

A targeted release portion can be added to the extended release system by means of either applying an immediate release layer on top of the extended release core; using coating or compression processes or in a multiple unit system such as a capsule containing extended and immediate release beads.

When used with respect to a pharmaceutical composition or other material, the term “sustained release” is art-recognized. For example, a therapeutic composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active salt and/or composition, for a sustained or extended period (as compared to the release from a bolus). This release may result in prolonged delivery of therapeutically effective amounts of any of the therapeutic agents disclosed herein.

Current efforts in the area of drug delivery include the development of targeted delivery in which the drug is only active in the target area of the body (for example, in cancerous tissues) and sustained release formulations in which the drug is released over a period of time in a controlled manner from a formulation. Types of sustained release formulations include liposomes, drug loaded biodegradable microspheres and drug polymer conjugates.

Delayed release dosage formulations are created by coating a solid dosage form with a film of a polymer which is insoluble in the acid environment of the stomach, but soluble in the neutral environment of the small intestines. The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Alternatively, a delayed release tablet may be formulated by dispersing tire drug within a matrix of a suitable material such as a hydrophilic polymer or a fatty compound. Suitable hydrophilic polymers include, but are not limited to, polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

A pulsed release-dosage is one that mimics a multiple dosing profile without repeated dosing and typically allows at least a twofold reduction in dosing frequency as compared to the drug presented as a conventional dosage form (e.g., as a solution or prompt drug-releasing, conventional solid dosage form). A pulsed release profile is characterized by a time period of no release (lag time) or reduced release followed by rapid drug release.

The phrases “parenteral administration” and “administered parenterally” as used herein refer to modes of administration other than enteral and topical administration, such as injections, and include without limitation intravenous, intramuscular, intrapleural, intravascular, intrapericardial, intra-arterial, intrathecal, intracapsular, intraorbital, intracardiac, intradennal, intraperitoneal, transtracheal, subcutaneous, subcuticular, intra-articular, subcapsular, subarachnoid, intraspinal and intrasternal injection and infusion.

Certain pharmaceutical compositions disclosed herein suitable for parenteral administration comprise one or more subject compositions in combination with one or more pharmaceutically acceptable sterile, isotonic, aqueous, or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic within the blood of the intended recipient or suspending or thickening agents.

### Methods and Materials

Preparation of Oligopeptide Solutions for Immunization:

The peptides were dissolved in 8M Urea to the concentration of 1.1 mg/ml. Peptides are dissolved at concentration 1.1 mg/ml. Conjugate Streptavidin-HRP (Str-HRP) as a carrier protein is dissolved in PBS at the concentration of 0.8 mg/ml. Peptide solution is mixed with conjugate Streptavidin-HRP to achieve standard final concentrations for peptides and conjugate.

Conjugate Streptavidin-PolyHRP20 (#SP20C) as a carrier protein was purchased from SDT (Germany), urea and salts were obtained from Fluka (Schweiz). All reagents were of analytical grade. All solutions were prepared using pyrogen free milliQ grade water. Dialysis was done with cellulose membrane D9777-100FT, Sigma (St. Louis, Mo.).

The following reference peptide was used with biotin: “SP-35” from gp41 env HIV-1 with the following amino acid sequence: H-Arg-Ile-Leu-Ala-Val-Glu-Arg-Tyr-Leu-Lys-Asp-Gln-Gln-Leu-Leu-Gly-Ile-Trp-Gly-Cys-Ser-Gly-Lys-Leu-Ile-Cys-Thr-Thr-Ala-Val-Pro-Trp-Asn-Ala-Ser-OH. Solutions of peptides were prepared into 3 ml glass vials ISO 8362-1 2R-CL-1 (Medical Glass, Bratislava, Slovakia) or PP Costar Microcentrifuge Tube (Cat.#3621, Corning Inc., USA) depend on the solution volume. Weighing was performed on balance R200D (Sartorius, Germany). Dispensing of solutions was conducted with Finnpipette, a digital adjustable volume pipettes 0.5-10 μl, 5-40 μl, 20-200 μl, 200-1000 μl, 1-5 ml.

Preparation of Conjugate Streptavidin-PolyHRP20 as a carrier protein: Conjugate Streptavidin-PolyHRP20 (Str-HRP 1 mg/ml) in solution containing 50% (v/v) glycerol. For removing of glycerol, Str-HRP was dialyzed against PBS. Volume of conjugate to increase after dialysis and was concentrated up to 1.25 (0.8 mg/ml) from initial volume.

Preparation of conjugate peptide+carrier protein: Peptides were dissolved in appropriate volume 8M Urea. After dissolving, 4 aliquots in 0.2 ml were taken from each peptide solution, mixed with 0.6 ml Str-HRP and incubated over night at +4° C. Conjugate peptide+carrier protein (Str-HRP) were frozen and stored at −20° C. until immunization. The final concentrations in peptide-Str-HRP solutions were 0.8 mg/ml and 0.6 mg/ml for peptide and Str-HRP, respectively. The final concentration of urea in peptide-Str-HRP solutions were 2M for all peptides.

Protocol of immunization (Rockland Immunochemicals, 2006-2007 Catalog, page 185):

Day 0: Immunization with complete Freund's adjuvant
Day 7: Booster with incomplete Freund's adjuvant
Day 14: Booster with incomplete Freund's adjuvant
Day 28: Booster with incomplete Freund's adjuvant
Day 38: Terminal bleeding of animals

Although Freunds's adjuvant is used for the mouse preparations for other mammal we might use adjuvants such as alum, calcium phosphate, aluminum salts (such as aluminum phosphate and/or aluminum hydroxide), tyrosine, liposomes, virosomes, emulsions (saponins), nanoparticles, microparticles, Iscoms and virus like particles.

Animals, materials and equipment: BALB/c female mouse, complete Freund's adjuvant (Calbiochem, USA), incomplete Freund's adjuvant (Calbiochem, USA), 2 ml syringe 22 G×1½″(BKMI, R. Korea), PP Costar Microcentrifuge Tube (Cat.#3621, Corning Inc., USA), Vortex Vibrofix VF1 (IKA-Werk, Germany), GP Centrifuge (Beckman, USA)

Immunization: Frozen 0.8 ml aliquots of peptide+Str-HRP conjugate (see above) were thawed at RT and mixed with 0.8 ml of appropriate adjuvant. The adjuvant was added and mixed on a vortex immediately before the injections. Immunization was performed by i.p. injections made with 100 μg peptide per animal in a final volume of 250 μl of 1:1 (v:v) peptide+Str-HRP:adjuvant.

Preparation of serum: After 38 days the mice were sacrificed. The blood was collected in 2-ml microcentrifuge tubes and allowed to clot at room temperature for about 1 hour. The microcentrifuge tubes were centrifuged with the clots for 15 min at 2500 g and the serum was then collected. The volume of each sample was no less 400 μl. Subsequently the samples were stored at −20° C.

Testing Immune Response to Individual Peptide:

Determination of mouse antibodies to peptide was based on indirect solid-phase immunoenzymatic assay with avidin on solid-phase. As test plates 96-well polystyrene plates high binding (#9018 Costar, USA) were used. All solution for ELISA: sample diluent (10 mM sodium phosphate, 500 mM NaCl, 0.5% BSA, 0.05% Tween-20, pH 7.4), conjugate diluent (10 mM sodium phosphate, 150 mM NaCl, 0.5% BSA, 0.05% Tween-20, pH 7.4), wash fluid (10 mM sodium phosphate, 300 mM NaCl, 0.05% Tween-20, pH 7.4), substrate buffer (50 mM sodium citrate and hydrogen peroxide, pH 5.0), TMB solution (3,3′,5,5′-tetramethylbenzidine), stop solution (2M sulphuric acid) were taken from EIA Kit for detection of antibody to HIV “Peptoscreen-2” (Amercard Ltd, Russia). The conjugate of rabbit antibody to mouse IgG with HRPO was conducted according to an inhouse procedure, and the avidin was prepared from egg white (Imtek, Russia). Dispensing of solutions was conducted with Finnpipette digital adjustable volume pipettes 0.5-10 μl, 5-40 μl, 20-200 μl, 200-1000 μl, 1-5 ml and 12-channel pipettes 5-50 μl, 50-200 μl. Information about additional technical equipment is the following: Plate washer ZLE201 (Amersham Inc., UK), incubator at 37° C.—Imperial II (Lab-Line Instruments Inc., USA), EIA plate reader—Luminometer-Photometer LM01A (Immunotech, Beckman Coulter Company, USA).

Peptides for binding on the avidin-coated plate were dissolved up to 2 mM in sample diluent immediately before the test procedure. EIA plates were coated by adding to the wells 100 of avidin dissolved 10 μg/ml in 50 mM carbonate buffer, at pH 9.5 and incubated for 20 h at 20° C. The plates were washed 4 times with washing fluid. Peptides 2 mM were diluted 100 μl/well and incubated for 60 min at 37° C. Control wells were incubated with avidin. The plates were washed 4 times with washing fluid. Serum from each mouse and negative control were diluted 1:100, 1:1000 and 1:10000 in sample diluent and was added to the wells, coated with corresponding peptide (100 μl per well) and incubated for 1 h at 37° C. The plates were then washed 4 times with washing fluid. Conjugates of rabbit anti-mouse IgG antibody with HRPO (dilution of 1:3000 in conjugate diluent) were added to the wells (100 μl per well). The plates were incubated for 0.5 h at 37° C. The plates were washed again 4 times with washing fluid. 100 of freshly prepared substrate solution (1 v TMB solution+7 v substrate buffer) were added to each well, and the plates were left at room temperature for 15 minutes in a dark place. A blue color developed in wells containing positive samples. Subsequently, 100 μl of stop solution were added to each well in the same sequence as the addition of substrate solution in step 3.3.7., turning the blue color into yellow. Absorbance reading of the plates was done within 50 minutes at 450 nm (A₄₅₀) using a plate reader.

Results:

The 11 oligopeptides tested fall into two groups:

a. Group of relatively strong immunogenicity (three peptides: ##6, 4 and 8);
b. Group of intermediate immunogenicity (eight peptides: ##2, 9, 11, 7, 1, 5, 10 and 3).

TABLE 1
The 11 oligopeptides with the highest antigenicity are listed
in the following table:

| | | | Antigenicity | |
|---|---|---|---|---|
| | | | compared to best | |
| | | | ranking peptide | |
| | | Antigenicity | (Average value | |
| Overall | | compared to | calculated by | |
| ranking | | best ranking | removing outlier | |
| of anti- | SEQ | peptide | which exceeded | From |
| genicity | ID# | (Median value) | 1 SD value) | Protein |
| 1 | 6 | 1.1 | 1.0 | Ag 85 |
| 2 | 4 | 1.0 | 1.4 | Ag 85 |
| 3 | 8 | 4.5 | 4.6 | Erp |
| 4 | 2 | 15.0 | 10.6 | HA |
| 5 | 9 | 22.3 | 16.1 | Erp |
| 6 | 11 | 34.2 | 16.5 | Hemolysine |
| 7 | 7 | 73.5 | 11.6 | Ag 85 |
| 8 | 1 | 38.1 | 44.5 | HA |
| 9 | 5 | 47.4 | 21.8 | Ag 85 |
| 10 | 10 | 24.5 | 27.6 | HA |
| 11 | 3 | 35.8 | 26.5 | Ag 85 |

The results show that the selected 11 oligopeptides show a variable degree of immunogenicity from intermediate (overall ranking #4 to #11) to relatively strong (ranking #1 to #3). These selected antigenicity inducing oligopeptides show a promise of an effective vaccine that may be produced for a mammalian vaccination use.

The antigenic epitopes of virulence-mediating proteins are, by definition, the sites were antibodies interfere with the virulence of a pathogen, in this invention against mycobacterium tuberculosis. The oligopeptides identified here represent such epitopes from no less than three virulence-mediating proteins of MTB. The preventive and/or therapeutic use of these oligopeptides as vaccines against tuberculosis represents a new potential in the global control of this epidemic.

In addition, it will be appreciated that the various peptide sequences, oligopeptide sequences, immunization processes, and methods of treatment disclosed herein may be embodied using means for achieving the various combinations of therapeutic dosage and delivery methods to treat a specific disease.

What is claimed is:

1. A immunogenic response generating composition, comprising:
a peptide sequence representing a virulence mediating protein for Mycobacterium Tuberculosis bacteria;
an adjuvant; and
a carrier protein.

2. The composition of claim 1, wherein the virulence mediating protein are at least one of a MT Heparin binding hemagglutinin, MT Antigen 85, MT exported repetitive protein and MT cytotoxin/hemolysin protein.

3. The composition of claim 2, wherein the virulence mediating protein MT Heparin binding hemagglutinin is at least one a SEQ ID 1 and SEQ ID 2.

4. The composition of claim 2, wherein the virulence mediating protein MT Antigen 85 is at least one a SEQ ID 3, SEQ ID 4, SEQ ID 5, SEQ ID 6 AND SEQ ID 7.

5. The composition of claim 2, wherein the virulence mediating protein MT exported repetitive protein is at least one of a SEQ ID 8 AND SEQ ID 9.

6. The composition of claim 2, wherein the virulence mediating protein and MT cytotoxin/hemolysin protein is at least one of a SEQ ID 10 AND SEQ ID 11.

7. The composition of claim 2, wherein the virulence mediating protein is at least one of SEQ ID 1 TO SEQ ID 11 and a combination of SEQ ID 1 to SEQ ID 11 thereof.

8. The composition of claim 1, wherein the adjuvant is an aluminum based salts such as an aluminum phosphate and/or aluminum hydroxide.

9. The composition of claim 1, wherein the adjuvant is at least one of a squalene, another suitable organic substance and virosome.

10. A method of immunizing a mammal to Mycobacterium Tuberculosis, the method comprising:
mixing a adjuvant and a carrier with an oligopeptide comprising at least one of a SEQ ID 1-11 and combination thereof to create a vaccine;
injecting said mammal with the vaccine to treat for tuberculosis infection.

11. A composition, comprising:
a peptide sequence representing a virulence mediating protein for Mycobacterium Tuberculosis bacteria; and
a carrier protein.

12. The composition of claim 11, further comprising:
an adjuvant to enhance the antigenicity in a mammal suffering from a Mycobacterium Tuberculosis infection.

13. The composition of claim 11, wherein the virulence mediating protein is at least one of a MT Heparin binding hemagglutinin, MT Antigen 85, MT exported repetitive protein and MT cytotoxin/hemolysin protein.

14. The composition of claim 11, wherein the virulence mediating protein is at least one of a MT Heparin binding hemagglutinin, MT Antigen 85, MT exported repetitive protein, MT cytotoxin/hemolysin protein and a combination thereof.

15. The composition of claim 14, wherein the virulence mediating protein MT Heparin binding hemagglutinin is at least one a SEQ ID 1 and SEQ ID 2.

16. The composition of claim 14, wherein the virulence mediating protein MT Antigen 85 is at least one a SEQ ID 3, SEQ ID 4, SEQ ID 5, SEQ ID 6 and SEQ ID 7.

17. The composition of claim 14, wherein the virulence mediating protein MT exported repetitive protein is at least one of a SEQ ID 8 and SEQ ID 9.

18. The composition of claim 14, wherein the virulence mediating protein MT cytotoxin/hemolysin protein is at least one of a SEQ ID 10 and SEQ ID 11.


SEQUENCE LISTING (11 sequences)

SEQ ID NO: 1 - protein, 14 aa, Artificial Sequence
  Mycobacterium Tuberculosis Synthetic Peptide
TDTRSRVEESRARL

SEQ ID NO: 2 - protein, 12 aa, Artificial Sequence
  Mycobacterium Tuberculosis Synthetic Peptide
SRYNELVERGEA

SEQ ID NO: 3 - protein, 12 aa, Artificial Sequence
  Mycobacterium Tuberculosis Synthetic Peptide
SMGRDIKVQFQG

SEQ ID NO: 4 - protein, 12 aa, Artificial Sequence
  Mycobacterium Tuberculosis Synthetic Peptide
LRAQDDYNGWDI

SEQ ID NO: 5 - protein, 14 aa, Artificial Sequence
  Mycobacterium Tuberculosis Synthetic Peptide
TYKWETFLTREMPA

SEQ ID NO: 6 - protein, 17 aa, Artificial Sequence
  Mycobacterium Tuberculosis Synthetic Peptide
SDPAWKRNDPMVQIPRL

SEQ ID NO: 7 - protein, 15 aa, Artificial Sequence
  Mycobacterium Tuberculosis Synthetic Peptide
GDNIPAKFLEGTLRT

SEQ ID NO: 8 - protein, 19 aa, Artificial Sequence
  Mycobacterium Tuberculosis Synthetic Peptide
VYESTETTERPEHHEFKQA

SEQ ID NO: 9 - protein, 15 aa, Artificial Sequence
  Mycobacterium Tuberculosis Synthetic Peptide
NELGASQAIDLLKGV

SEQ ID NO: 10 - protein, 12 aa, Artificial Sequence
  Mycobacterium Tuberculosis Synthetic Peptide
TDSERAWVSRGA

SEQ ID NO: 11 - protein, 9 aa, Artificial Sequence
  Mycobacterium Tuberculosis Synthetic Peptide
AGRRCLDAG